(19) United States
(12) Patent Application Publication
(10) Pub. No.: US 2025/0344723 A1
(43) Pub. Date: Nov. 13, 2025

(54) METHOD OF FEEDING (PRE)RUMINANTS

(71) Applicant: Nutreco IP Assets B.V., Boxmeer (NL)

(72) Inventors: David Manuel BRAVO, Boxmeer (NL); Emma WALL, Boxmeer (NL); Yanming HAN, Boxmeer (NL)

(21) Appl. No.: 19/279,793

(22) Filed: Jul. 24, 2025

Related U.S. Application Data
(63) Continuation of application No. PCT/EP2024/051680, filed on Jan. 24, 2024.

(30) Foreign Application Priority Data
Jan. 25, 2023 (IN) 202341005083

(51) Int. Cl. A23K 10/30; A23K 20/147; A23K 20/158; A23K 20/163; A23K 50/10; A23K 50/60

(52) U.S. Cl. CPC A23K 10/30; A23K 20/147; A23K 20/158; A23K 20/163; A23K 50/10; A23K 50/60

(57) ABSTRACT

The present invention provides a method of feeding a pre-ruminant or ruminant animal, said method comprising the step of administering to said animal a herbal oil/extract composition. Moreover, the invention provides a composition comprising such herbal oil/extract composition. Also, the invention provides a method of preventing and/or treating parasitic infections and diseases caused thereby such as cryptosporidiosis using such herbal oil/extract composition.

### CROSS-REFERENCE TO RELATED APPLICATIONS

This application is a continuation of International Application No. PCT/EP2024/051680 filed Jan. 24, 2024, which application claims priority to Indian Patent Application number 202341005083 filed Jan. 25, 2023, the entire contents of all of which are hereby incorporated by reference herein.

### FIELD OF THE INVENTION

The present disclosure describes botanical composition(s) comprising standardized Cinnamon extract or oil, Tea and/or Marigold extract and/or Pomegranate and/or Tamarind extract, as an additive/supplement in livestock feed/water/milk/milk replacers, pet food/milk/milk replacers/water and/or human application by administering directly as a nutraceutical supplement or added as an additive/supplement in food/water, to improve gut health, prevent gut infections, improve disease resistance, strengthen gut integrity and immunity and alleviate ill effects of gut infection and help improve general health and performance. The present invention more particularly relates to compositions comprising at least two of these extracts in the bioavailable, fat encapsulated and/or water soluble formulations, or by simply blending together with a carrier to serve the above mentioned purpose.

### BACKGROUND OF THE INVENTION

During their lifetime, ruminants become infected with gastrointestinal (GI) nematodes. The severity of the resulting disease can range from no clinical signs to debilitating, fatal disease, depending on the strength of the animals' acquired immunity, the magnitude of exposure, and concurrent stressors or illness.

Ruminants acquire protective, non-sterilizing immunity to GI nematodes over time, after exposure to the parasites. Therefore, young, growing animals are the most susceptible to disease. Although the presence of nematode-specific antibodies has been shown in the milk and colostrum of cattle and small ruminants, it is not known whether these antibodies induce protective immunity. Animals kept in an environment where they are not exposed to parasites do not develop protective immunity, and they suffer severe clinical signs, even as adults, on first exposure.

Other factors that affect the presence of clinical signs of GI parasitism include stressors or illnesses that may impair the immune system response. Examples include late pregnancy, lactation, weaning, shearing, overcrowding, inadequate nutrition, and primary illnesses. A periparturient rise in egg counts of parasites occurs for most GI parasites during late pregnancy and early lactation. This periparturient rise can represent an additional source of infection for neonates, or increased pasture contamination for herd mates.

Cryptosporidium is a microscopic parasite that causes the diarrheal disease cryptosporidiosis. Calves affected with Cryptosporidium are usually one to four weeks of age. These calves become weak and lethargic and present with loose to watery stool that may be mild or severe in intensity. Faeces can contain mucus, blood, undigested milk, or bile. Tenesmus (straining to defecate) may be seen. Oocyst shedding typically begins with diarrhea and continues for a few days passed resolution of clinical signs. There are many species of Cryptosporidium that infect animals, some of which also infect humans. The parasite is protected by an outer shell that allows it to survive outside the body for long periods of time and makes it very tolerant to chlorine disinfection.

There is no affective or approved treatment for Cryptosporidiosis. Morbidity is high with this disease, but mortality is generally low. However, calves do need intensive supportive care. They need fluid therapy to counteract and prevent further dehydration as well as electrolytes to replace those lost due to diarrhoea. They also need nutritional support to give them energy to fight disease and repair their bodies. Calves that suffer from cryptosporidiosis usually have a slower growth rate than other calves, that often persists into later life. Also, it is more labor-intensive to support these calves through their disease. Also, farmers have to purchase rehydration fluids. Another parasitic disease of livestock is coccidiosis. Coccidiosis is a parasitic disease of the intestinal tract of poultry that is caused by protozoan parasites of the genus Eimeria. Coccidiosis is of worldwide occurrence and every year it costs the livestock and pet industry many millions of dollars to control. Coccidiosis is caused by different species of Eimeria and each species has its own specificity towards particular gut region. Similarly, pathogens such as enteric pathogenic bacteria, parasites and virus cause serious infections in humans, affecting quality of life and general health. All these pathogens cause localized damage to the gut, while it utilizes host machinery for its multiplication/propagation. This leads to disruption of gut integrity, digestive functions and immune responses and also leads to on-set of inflammatory reactions. This also paves way for secondary pathogens, such as Clostridium perfringens to cause secondary infection leading to necrotic enteritis. All these primary and secondary infections lead to heavy production loss and mortality in livestock's and affects general health and immunity in the case of humans.

There is ample literature available on the strategies to improve gut health which includes, but not limited to the use of prebiotics, probiotics, postbiotics, organic acids and botanicals and few of such strategies are discussed herein below.

EP3981412 covers composition(s) containing prebiotics extracted from beetroot or from milk or from garlic, suitable for the preventative treatment of intestinal infections such as clostridiosis, intestinal parasitosis, cryptosporidiosis and coccidiosis, as well as infections caused by some types of E. coli bacteria, and metabolic diseases such as pre-eclampsia.

CN109480087 covers an anti-cococidal formulation, which comprises sweet wormwood, hairy vein agrimony, fleece-flower root, Chinese bulbul and cortex cinnamomi.

US20160165945 discloses a composition for providing antimicrobial properties to food which comprises grapefruit and/or grapefruit seed extract and citrus fruit extract(s).

CN113648345 describes composition for Preventing and Treating Coccidiosis which comprises pomegranate extract; Spanish needles herb extract; Acalypha australis extract; Herba Pteridis multifidae extract; Herba Portulacae extract; blumea oil and tea tree essential oil.

JP2001354580 discloses a composition containing oregano, sage, rosemary, nutmeg, ginger, cinnamon and clove.

Although the above prior arts suggest the use of prebiotics, probiotics, post-biotics, either alone or in combination with botanicals for providing antimicrobial properties to prevent Coccidiosis in livestock; however, none of them discuss the host mediated responses such as improving gut integrity, disease resistance and muscle health. The present invention discuss the use of said botanicals alone or in combination improves the above host responses and also act as anti-inflammatory and antimicrobial, so as to provide complete gut solution to the host-livestock, pet and humans

Also, none of the prior art provides the use of formulations in the form of bioavailable, fat encapsulated and/or water soluble formulations using botanicals, for improving gut health, e.g., in pre-ruminants or ruminants.

In the light of the foregoing, there remains a need in the art for the provision of effective and safer feed supplements, that prevents the spread of pathogenic infections, improves not only the quality of life, disease resistance, gut integrity, performance and general & immune health of livestock and other hosts, but also assures ultimate safety for livestock, pet and human consumption, which becomes the objective of the present invention.

It is also an object of the present invention to provide a composition capable of preventing and/or treating parasitic infections in pre-ruminants or ruminants, in particular, infections by Cryptosporidium parvum in calves. Moreover, it is an object of the present invention to provide a composition capable of preventing and/or treating cryptosporidiosis.

### SUMMARY OF THE INVENTION

In pursuit of the above objective(s), the present disclosure describes compositions comprising at least two extracts selected from standardized 1) Cinnamon extract or oil, 2) Tea and/or Marigold extract and/or 3) Pomegranate and/or Tamarind extract, as an additive/supplement in livestock feed/water/milk/milk replacers, pet food/milk/milk replacers/water and/or human application by administering directly as a nutraceutical supplement or added as an additive/supplement in food and/or water, to improve gut health, prevent gut infections, improve disease resistance, strengthen gut integrity and immunity and alleviate ill effects of gut infection and to improve or maintain general health and/or performance.

In another aspect, the disclosure provides composition comprises at least two of 1) Cinnamon extract or oil, 2) Tea and/or Marigold extract and/or 3) Pomegranate and/or Tamarind extract, in bioavailable, fat encapsulated and/or water soluble formulations, or by simply blending together with a carrier to serve the above mentioned purpose.

In an aspect, the present invention provides a method of feeding a pre-ruminant or ruminant animal, said method comprising the step of administering to said animal a composition comprising cinnamon extract or oil, tea extract and pomegranate extract. The composition may be comprised in an aqueous solution, e.g., water such as drinking water, or a milk replacer. Alternatively, the composition may be comprised in a pre-ruminant or ruminant feed composition. The animal may be a calf or a dairy or beef cow.

In another aspect, the present invention provides a composition, said composition preferably being a milk replacer, comprising:
15-30 wt %, based on dry matter content, fats;
15-35 wt % based on dry matter content milk protein;
35-55 wt % based on dry matter content sugars, said sugars preferably comprising lactose;
said composition further comprising a composition comprising cinnamon extract or oil, tea extract and pomegranate extract.

The milk replacer composition may be in powder form or in aqueous form after reconstitution of a powder in water.

The milk replacer composition may comprise less than about 5 wt % based on dry matter content starch and/or less than about 10 wt % based on dry matter content ash.

The fats in the milk replacer composition may comprise or consist of vegetable fats. The vegetable fats may comprise palm oil, coconut oil, palm kernel oil, rapeseed oil, linseed oil, sunflower oil, MCT oil, and the like.

The milk protein may comprises or consists of casein, and/or the milk protein may comprise one or more of β-lactoglobulin, α-lactalbumin, blood serum albumin, immunoglobulins, lactoferrin, and transferrin.

The sugars comprised in the milk replacer composition may comprise lactose.

In another aspect, the present invention provides a method of preventing, reducing the severity of, and/or treating cryptosporidiosis, said method comprising the step of administering to a subject a composition comprising cinnamon extract or oil, tea extract and pomegranate extract.

In a further aspect, the present invention provides a method of preventing, reducing the severity of, and/or treating parasitic infections in a preruminant and/or ruminant animal, said method comprising the step of administering to preruminant and/or ruminant animal a composition comprising cinnamon extract or oil, tea extract and pomegranate extract.

### General Definitions

In the following description and examples, a number of terms are used. In order to provide a clear and consistent understanding of the specification and claims, including the scope to be given to such terms, the following definitions are provided. Unless otherwise defined herein, all technical and scientific terms used have the same meaning as commonly understood by one of ordinary skill in the art to which this invention belongs. The disclosures of all publications, patent applications, patents and other references cited herein are incorporated herein in their entirety by reference.

The term “extract” as used herein refers to an extract of a plant or plant part, or any solids extracted from a plant or plant part. The solids extracted may be comprised in the original extract or a concentrated form thereof, or may be dried and optionally reconstituted in another solvent. The term ‘about’, as used herein indicates a range of normal tolerance in the art, for example within 2 standard deviations of the mean. The term “about” can be understood as encompassing values that deviate at most 10%, 9%, 8%, 7%, 6%, 5%, 4%, 3%, 2%, 1%, 0.5%, 0.1%, 0.05%, or 0.01% of the indicated value.

The terms “comprising” or “to comprise” and their conjugations, as used herein, refer to a situation wherein said terms are used in their non-limiting sense to mean that items following the word are included, but items not specifically mentioned are not excluded. It also encompasses the more limiting verb “to consist essentially of” and “to consist of”.

Reference to an element by the indefinite article “a” or “an” does not exclude the possibility that more than one of the elements is present, unless the context clearly requires that there be one and only one of the elements. The indefinite article “a” or “an” thus usually means “at least one”.

### DETAILED DESCRIPTION OF THE INVENTION

This patent application has been drafted into sections. However, these sections should not be read in isolation. Unless otherwise specified, each section is to be read in combination with the other sections. The various optional and preferred features can also be combined, even when taken from different parts of the specification. Likewise, all “aspects” and “embodiments” can be combined. No separation of embodiments is intended, unless explicitly stated.

### Compositions

Considering the pathophysiology and need for botanical alternative to tackle the issues pertaining to gut health, the present inventors have aimed at developing holistic product(s) which can improve performance, reduce gut inflammation and improve gut integrity, prevent gut infections, improve disease resistance, strengthen immunity and alleviate ill effects of gut infection and help improve general health and performance in the livestock, pet and humans. With this objective in mind, in-vitro and functional (cell line studies) assays were conducted to screen and identify five top performing botanicals, viz., 1) Cinnamon extract; 2) Tea and/or marigold extracts and 3) Pomegranate and/or tamarind extract, to address the issues of maintaining gut health, improving disease resistance and development or growth of the muscles. These botanicals have unique properties. Cinnamon extract acts as a strong anti-inflammatory agent that helps to recover from the damage caused by enteric pathogens such as Eimeria and Clostridia in gut. Tea and/or marigold extracts are found to be effective in improving the gut integrity there by improving the immune responses, growth and hence strengthens disease resistance. Pomegranate and/or tamarind extract was found to influence factors that are responsible for muscle development/growth.

Accordingly, the present disclosure describes composition(s), comprising at least two extracts selected from standardized Cinnamon extract or oil, Tea and/or Marigold extract and/or Pomegranate and/or Tamarind extract formulated as a bioavailable, fat encapsulated and/or water-soluble composition or blends, for use as an additive/supplement in livestock feed/milk/milk replacer/water, pet food/milk/milk replacers/water and/or direct human nutraceutical supplement, or use in human food/milk replacers/water to improve gut health, prevent gut infections, improve disease resistance, strengthen immunity and alleviate ill effects of gut infection and help improve general health and performance.

The fats that can be used to encapsulate the botanicals of the invention may be selected from the group consisting of, but not limited to, Glycerol Mono stearate, Hydrogenated palm stearin, Hydrogenated rapeseed oil and other vegetable oils or fats.

In certain embodiments, Maltodextrin is used as a water soluble carrier.

In certain embodiments, the bioavailable, fat encapsulated or water soluble blends or compositions may be provided to the livestock by mixing with the feed in specific concentrations anywhere between 10 ppm to 1500 ppm, such as between 15 ppm and 1400 ppm, between 20 ppm and 1300 ppm, between 25 ppm and 1200 ppm, between 30 ppm, and 1100 ppm, between 35 ppm and 1000 ppm, between 40 ppm and 900 ppm, between 45 ppm and 800 ppm, or between 50 ppm and 750 ppm.

In certain embodiments, the blends, bioavailable, fat encapsulated or water soluble compositions may be provided to the pets by mixing with the food/water/milk/milk replacers in specific concentrations anywhere between 10 ppm to 1500 ppm, such as between 15 ppm and 1400 ppm, between 20 ppm and 1300 ppm, between 25 ppm and 1200 ppm, between 30 ppm, and 1100 ppm, between 35 ppm and 1000 ppm, between 40 ppm and 900 ppm, between 45 ppm and 800 ppm, or between 50 ppm and 750 ppm.

In certain embodiments, the blends, bioavailable, fat encapsulated or water soluble compositions may be supplemented to humans directly or by mixing with the food/water/milk/milk replacers in specific concentrations anywhere between 10 mg to 1500 mg.

In certain embodiments, the water soluble carriers that can be used to formulate water soluble composition using botanicals of the invention are selected from the carriers group consisting of, but not limited to, Maltodextrin, Gum acacia, Lactose, Dextrans, etc.

The Tea extract as referred herein may be a black tea extract or a green tea extract. In certain embodiments, the Tea extract as referred herein is the combination of green tea extract and black tea extract.

Accordingly, in one embodiment, the invention provides a fat encapsulated composition comprising;
a) Cinnamon extract or oil in an amount of 0.1 to 20%;
b) Pomegranate and/or Tamarind extract in an amount of 0.1 to 20% and/or;
c) Tea and/or Marigold extract in an amount of 0.1 to 20%; and
d) Fats in an amount of 80 to 98%.

The fats that can be used to encapsulate the botanicals of the invention may be selected from the group consisting of, but not limited to, Glycerol Mono stearate, Hydrogenated palm stearin, Hydrogenated rapeseed oil and other vegetable oils or fats. Said vegetable oils or fats may be partly or fully hydrogenated. In a suitable embodiment, said fats are rumen inert, i.e., are not or hardly degraded in the rumen.

According to another embodiment, the invention provides a water soluble composition comprising;
a) Cinnamon extract or oil in an amount of 0.1 to 20%;
b) Pomegranate and/or Tamarind extract in an amount of 0.1 to 20%; and/or
c) Tea and/or Marigold extract in an amount of 0.1 to 20%; and
d) Water soluble carriers in an amount of 50 to 90%.

The water soluble carriers may be selected from the group consisting of, but not limited to, Maltodextrin, Gum acacia, Lactose, Dextrans etc. In a suitable embodiment, the water-soluble carrier is maltodextrin.

According to yet another embodiment, the invention provides a composition comprising:
a) Cinnamon extract or oil in an amount of 0.1 to 20%;
b) Pomegranate and/or Tamarind extract in an amount of 0.1 to 20%;
c) Tea and/or Marigold extract in an amount of 0.1 to 20%;
d) Maltodextrin in an amount of 50 to 90%; and
e) Emulsifier in an amount of 0.1 to 20%.

In a preferred embodiment, the disclosure provides a composition comprising:
a) Cinnamon extract or oil in an amount of 0.1 to 20%;
b) Pomegranate extract in an amount of 0.1 to 20%;
c) Tea extract in an amount of 0.1 to 20%;
d) Maltodextrin in an amount of 50 to 90%; and
e) Emulsifier in an amount of 0.1 to 20%.

In the above embodiment, Maltodextrin, Cyclodextrin, lactose, Gum acacia etc. may be used to produce water soluble composition. The emulsifiers may be selected from the group consisting of, but not limited to, PEG, propylene glycol, glycerol, Polysorbate 20, Polysorbate 80, lecithin, SLS/other gums and the like.

In an embodiment, the proposed dosage of the composition may be in the range of 10 to 1500, such as in the range of 5 and 1400, of 20 and 1300, of 25 and 1200, of 30 and 1100, of 35 and 1000, of 40 and 900, of 45 and 800, or of 50 and 750, gram/ton of animal feed.

In another embodiment, the proposed dosage of the composition in humans may be in the range of 10 mg to 1500 mg/day.

In yet another embodiment, the proposed dosage of the composition shall be between 10 to 1500 ppm in pet food.

### Milk Replacer Composition

The composition taught herein may be used in a milk replacer, preferably a calf milk replacer (CMR).

In an aspect, the present disclosure provides a (milk replacer) composition comprising 15-30 wt %, based on dry matter content, fats; 15-35 wt % based on dry matter content milk protein; 35-55 wt % based on dry matter content sugars, said sugars preferably comprising lactose; said composition further comprising a composition comprising cinnamon extract or oil, tea extract and pomegranate extract as taught herein.

Said composition is suitable to be used as a milk replacer for calves.

Calf milk replacers (CMRs) provide a convenient way to feed non-human young mammals, such as pre-ruminant calves. They can be stored long term as powder and mixed with water just prior to feeding. Calves can then be milk reared anywhere and at any time without having to source liquid whole milk. Thus, the milk replacer composition taught herein may be in powder form, or may be in aqueous form after reconstitution of the powder in water. The commercial product is usually a powder that is suitable for reconstitution in water on-farm.

Milk replacers may be formulated from by-products of dairy processing, together with animal and/or vegetable fats plus added vitamins and minerals. The by-product of butter making is skim milk, which consists mainly of lactose and all the milk proteins. However, it has only half the energy value of whole milk. Whey, the by-product of cheese making, consists only of lactose, albumin and globulin, and is even lower in nutritive value. When these dairy processing by-products are used as the basis of milk replacers, additional fats are required.

The composition taught herein may comprise 15-30 wt %, such as 15-28 wt %, 15-27 wt %, or 15-26 wt %, preferably 16-24 wt %, more preferably 17-23 wt %, even more preferably 18-22 wt %, based on dry matter content, fat. Said fat may comprise animal fats and/or vegetable fats. In an embodiment, the fat comprises vegetable fats. Suitable vegetable fats include, without limitation, palm oil, coconut oil, palm kernel oil, rapeseed oil, linseed oil, sunflower oil, MCT oil, and the like. Suitable animal fats include, without limitation, tallow, lard, and insect fats. The composition taught herein may further comprise 15-35 wt %, such as 16-33 wt %, 17-30 wt %, or 18-28 wt %, preferably 19-27 wt %, more preferably 20-26 wt %, or 20-25 wt %, based on dry matter content, protein. Said protein preferably comprises proteins of milk origin (“milk proteins”), such as those from whey, skim milk and buttermilk powders. Spray-dried milk powder or whey powder are the preferred source of powder for milk replacers. In an embodiment, the milk protein comprises or consists of casein. In an embodiment, the milk protein comprises one or more of β-lactoglobulin, α-lactalbumin, blood serum albumin, immunoglobulins, lactoferrin, and transferrin.

The composition further comprises 35-55 wt %, based on dry matter content, sugars, said sugars preferably comprising or consisting of lactose, preferably at least partially derived from dairy products by-processing.

In an embodiment, the composition comprises less than about 5 wt % based on dry matter content starch and/or less than about 10 wt % based on dry matter content ash.

In an embodiment, the composition comprises an antioxidant to reduce the deterioration of fat during storage.

Particularly when in powder form, long-term storage of CMR powders may be important. Hence, they may be packaged properly to keep out air and moisture. They may be vacuum sealed in a plastic bag then enclosed in a light-proof bag.

The milk replacer composition taught herein may be provided with written instructions on how to reconstitute the milk replacer composition in powder form in water to obtain the milk replacer composition taught herein in aqueous form. Additionally, the written instructions may comprise further information on amount of feeding and/or frequency of feeding. Hence, the milk replacer composition taught herein may be used as a milk replacer for calves.

### Animal Feed Composition

The present disclosure provides an animal feed comprising the composition taught herein. The animal feed may be in any form suitable for ingestion by the relevant animal. The animal feed generally comprises protein, fat, carbohydrates, minerals and vitamins. It may further comprise antioxidants, pigments, organic acids, enzymes, phytogenic compounds, and/or other feed additives. The skilled person knows how to formulate a suitable animal feed depending on the target animal, life stage of the target animal, and/or physical condition of the target animal.

In an embodiment, the animal feed taught herein may be a feed suitable for ingestion by pre-ruminants such as calves or ruminants such as dairy cows, beef cattle, sheep, goats, and the like. The skilled person knows how to formulate a feed suitable for the particular species of pre-ruminants or ruminants as well as the life stage of the particular species of pre-ruminants or ruminants.

In an embodiment, the proposed dosage of the composition in the animal feed may be in the range of 10 to 1500, such as in the range of 5 and 1400, of 20 and 1300, of 25 and 1200, of 30 and 1100, of 35 and 1000, of 40 and 900, of 45 and 800, or of 50 and 750, gram/ton of animal feed.

### Methods/Uses

The compositions taught herein are expected to accomplish the following,
1. Controls pathogen shedding/spread of infection
2. Resistance to parasitic infections like Eimeria and Coccidiosis
3. Resistance to Bacterial infections with E. coli, Salmonella, Shigella
4. Improves general health and performance
5. Improves gut integrity
6. Improves muscle development
7. Reduction in inflammation
8. Improve immunity
9. Improve gut health

The present disclosure provides a method of feeding a pre-ruminant or ruminant animal, said method comprising the step of administering to said animal a composition comprising cinnamon extract or oil, tea extract and pomegranate extract, or a composition as taught herein. In an embodiment, said pre-ruminant animal is a calf. In another embodiment, said ruminant animal is a dairy or beef cow.

The composition may be a milk replacer composition as taught herein, or may be an animal feed suitable for pre-ruminant animals such as calves or ruminant animals such as cows.

The present disclosure also provides a method of preventing, reducing the severity of, and/or treating cryptosporidiosis in a subject, e.g., in a pre-ruminant and/or ruminant animal, said method comprising the step of administering to said subject a composition comprising cinnamon extract or oil, tea extract and pomegranate extract, or a composition as taught herein.

A subject as referred to herein may be any subject, but is preferably a farming animal or a companion animal, more preferably a pre-ruminant or ruminant animal.

The present disclosure further provides a method of preventing, reducing the severity of, and/or treating microbial infections, such as infections by bacteria, yeast, fungi or parasites, in a pre-ruminant and/or ruminant animal, said method comprising the step of administering to said animal a composition comprising cinnamon extract or oil, tea extract and pomegranate extract, or a composition as taught herein.

The present disclosure further provides a method of preventing, reducing the severity of, and/or treating parasitic infections in a pre-ruminant and/or ruminant animal, said method comprising the step of administering to said animal a composition comprising cinnamon extract or oil, tea extract and pomegranate extract, or a composition as taught herein.

The pre-ruminant or ruminant animal may be any known pre-ruminant or ruminant animals, e.g., selected from the group consisting of a dairy calf or cow, a beef calf or cow, a veal calf or cow, a sheep lamb or sheep, a goat kid or goat, a buffalo calf or buffalo, a moose calf or moose, and elk calf or elk, or a bison calf or bison, but is preferably a dairy calf or cow, a beef calf or cow, a veal calf or cow.

The parasitic infection may be caused by any type of parasite known to affect pre-ruminant or All grazing animals become infected with GI nematodes during their lifetime. The severity of the resulting disease can range from no clinical signs to debilitating, fatal disease, depending on the strength of the animals' acquired immunity, the magnitude of exposure, and concurrent stressors or illness.

Ruminants acquire protective, non-sterilizing immunity to GI nematodes over time, after exposure to the parasites. Therefore, young, growing animals are the most susceptible to disease. Although the presence of nematode-specific antibodies has been shown in the milk and colostrum of cattle and small ruminants, it is not known whether these antibodies induce protective immunity. Animals kept in an environment where they are not exposed to parasites do not develop protective immunity, and they suffer severe clinical signs, even as adults, on first exposure. ruminant animals. Cryptosporidiosis is one of most prevalent parasitic infections in calves. Non-limiting examples of parasites that may infect ruminants include Paramphistomum spp (Flukes), Haemonchus spp (Barber's Pole Worm, Large Stomach Worm, Wire Worm), Ostertagia ostertagi (Medium or Brown Stomach Worm), Trichostrongylus axei (Small Stomach Worm), Mecistocirrus digitatus, Cooperia spp (Bankrupt Worm, Small Intestinal Worm), Trichostrongylus spp (Bankrupt Worms), Strongyloides papillosus (Intestinal Threadworm), Bunostomum phlebotomum (Hookworm), Nematodirus spp (Thread-Necked Intestinal Worms), Toxocara vitulorum (Roundworm), Moniezia spp (Tapeworms), Aonchotheca (formerly Capillaria) bovis, Oesophagostomum spp (Nodular Worms), Chabertia ovina (Large-Mouth Bowel Worm), and Trichuris spp (Whipworm).

The present disclosure also provides a method of preventing, reducing the severity of, and/or treating diarrhea in a pre-ruminant and/or ruminant animal, said method comprising the step of administering to said pre-ruminant and/or ruminant animal a composition comprising cinnamon extract or oil, tea extract and pomegranate extract, or a composition as taught herein. In an embodiment, the diarrhea is caused by an infection, such as a parasite infection, e.g., by infection with a Cryptosporidium species, e.g., Cryptosporidium parvum.

The present disclosure further provides a method of reducing oocyst shedding in a subject, said method comprising the step of administering to said subject a composition comprising cinnamon extract or oil, tea extract and pomegranate extract, or a composition as taught herein. In an embodiment, the oocyst is a C. parvum oocyst.

Further, the present disclosure provides a method of maintaining gut integrity and/or gut structure in a subject upon infection of said subject, said method comprising the step of administering to said subject a composition comprising cinnamon extract or oil, tea extract and pomegranate extract, or a composition as taught herein.

Additionally, the present disclosure provides a method of preventing or reducing body weight loss in a subject due to an infection, said method comprising the step of administering to said subject a composition comprising cinnamon extract or oil, tea extract and pomegranate extract, or a composition as taught herein. In an embodiment, the body weight loss in said subject is caused by an infection, such as a parasite infection, e.g., by infection with a Cryptosporidium species, e.g., Cryptosporidium parvum.

In an embodiment, the benefits described herein obtained in animals fed the compositions as taught herein are relative to results obtained in subjects/animals not fed the compositions as taught herein, or may be relevant to non-diseased and/or non-infected subject/animals.

The present invention is further illustrated, but not limited, by the following examples. From the above discussion and the examples, one skilled in the art can ascertain the essential characteristics of the present invention, and without departing from the teaching and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Exemplary Compositions

Composition 1

| SI. | | Quantity | |
|---|---|---|---|
| No. | Ingredients | % range | Function |
| 1 | Cinnamon extract /oil | 0.1 to 20% | Active Ingredient |
| 2 | Pomegranate/ Tamarind extract | 0.1 to 20% | Active Ingredient |
| 3 | Tea / Marigold extract | 0.1 to 20% | Active Ingredient |
| | Glycerol Mono stearate/ | | Fat used for |
| 4 | Hydrogenated palm stearin/ | 40-99% | encapsulation |
| | Hydrogenated rapeseed oil/ | | |
| | Other hydrogenated vegetable | | |
| | oil or fats | | |

The composition is encapsulated in vegetable fat as carrier.

Composition 2:

| SI. | | Quantity | |
|---|---|---|---|
| No. | Ingredients | % range | Function |
| 1 | Cinnamon extract /oil | 0.1 to 20% | Active Ingredient |
| 2 | Pomegranate/ Tamarind extract | 0.1 to 20% | Active Ingredient |
| 3 | Tea / Marigold extract | 0.1 to 20% | Active Ingredient |
| 4 | Maltodextrin/Gum acacia/ | 40-99% | Water soluble |
| | Lactose/ Dextrans | | carriers used |
| | | | for encapsulation |

The composition is encapsulated in water soluble carrier.

Composition 3

| SI. | | Quantity | |
|---|---|---|---|
| No. | Ingredients | % range | Function |
| 1 | Cinnamon extract /oil | 0.1 to 20% | Active |
| | | | Ingredient |
| 2 | Pomegranate/ Tamarind extract | 0.1 to 20% | Active |
| | | | Ingredient |
| 3 | Tea / Marigold extract | 0.1 to 20% | Active |
| | | | Ingredient |
| 4 | Maltodextrin/Lactose/Sucrose/Cyclo- | 40-99% | Water |
| | dextrin/ | | Soluble |
| | Other water soluble carriers | | carrier |
| 5 | Polysorbate/ other | 1-20% | |
| | emulsifier/other gums | | |

Composition 4:

| SI. | | Quantity | |
|---|---|---|---|
| No. | Ingredients | % range | Function |
| 1 | Cinnamon extract /oil | 0.1 to 20% | Active Ingredient |
| 2 | Pomegranate extract | 0.1 to 20% | Active Ingredient |
| 3 | Tea extract | 0.1 to 20% | Active Ingredient |
| 4 | Glycerol Mono stearate/ | 40-99% | Fat used for |
| | Hydrogenated vegetable | | encapsulation |
| | fats/Hydrogenated rapeseed | | |
| | oil/Other vegetable oils | | |
| 5 | Vegetable oil (optional) | 0% to Upto 30% | |
| 6 | Emulsifiers (lecithin, | 0% to up to 30% | |
| | polysorbates, etc) | | |
| | (Optional) | | |

Composition 5:

| SI. | | Quantity | |
|---|---|---|---|
| No. | Ingredients | % range | Function |
| 1 | Cinnamon extract /oil | 0.1 to 20% | Active Ingredient |
| 2 | Pomegranate extract | 0.1 to 20% | Active Ingredient |
| 3 | Tea extract | 0.1 to 20% | Active Ingredient |
| 4 | Maltodextrin/Cyclo- | 75-89% | Used for |
| | dextrin/Lactose | | encapsulation |
| 5 | Polysorbate/ other emulsifier | 1-20% | |
| | natural or synthetic/other gums | | |

Composition 6:

| SI. | | Quantity | |
|---|---|---|---|
| No. | Ingredients | % range | Function |
| 1 | Cinnamon extract /oil | 0.1 to 20% | Active Ingredient |
| 2 | Pomegranate extract | 0.1 to 20% | Active Ingredient |
| 3 | Tea extract | 0.1 to 20% | Active Ingredient |
| 4 | Hydrogenated palm stearin/ | 40-99% | Fat used for |
| | Hydrogenated rapeseed oil/ | | encapsulation |
| | Other vegetable oil | | |

Composition 7:

| SI. | | Quantity | |
|---|---|---|---|
| No. | Ingredients | % range | Function |
| 1 | Cinnamon extract /oil | 0.1 to 20% | Active Ingredient |
| 2 | Pomegranate extract | 0.1 to 20% | Active Ingredient |
| 3 | Tea extract | 0.1 to 20% | Active Ingredient |
| 5 | Polysorbate/propylene | 1-99% | |
| | glycol/glycerol/lecithin/phospho- | | |
| | tidyl choline | | |

Composition 8

| SI. | | Quantity | |
|---|---|---|---|
| No. | Ingredients | % range | Function |
| 1 | Cinnamon extract /oil | 0.1 to 20% | Active Ingredient |
| 2 | Pomegranate/ Tamarind extract | 0.1 to 20% | Active Ingredient |
| 3 | Tea / Marigold extract | 0.1 to 20% | Active Ingredient |
| 4 | Carriers/Inactive materials | 40-99% | Fat used for |
| | | | encapsulation |

Composition 9

| SI. | | Quantity | |
|---|---|---|---|
| No. | Ingredients | % range | Function |
| 1 | Cinnamon extract /oil | 0.1 to 20% | Active Ingredient |
| 2 | Tea/marigold extract | 0.1 to 20% | Active Ingredient |
| | Carriers/Inactive materials/ | | Fat used for |
| | Hydrogenated palm stearin/ | | encapsulation or |
| 3 | Hydrogenated rapeseed oil/ | 20-99% | water soluble |
| | Other vegetable oil | | carriers used |
| | Maltodextrin/Gum | | for encapsulation |
| | acacia / Lactose/ Dextrans | | |

Composition 10

| SI. | | Quantity | |
|---|---|---|---|
| No. | Ingredients | % range | Function |
| 1 | Cinnamon | 0.1 to 20% | Active Ingredient |
| 2 | Pomegranate/ Tamarind extract | 0.1 to 20% | Active Ingredient |
| 3 | Carriers/Inactive materials/ | 20-99% | Fat used for |
| | Hydrogenated palm stearin/ | | encapsulation or |
| | Hydrogenated rapeseed oil/ | | water soluble |
| | Other vegetable oil | | carriers used |
| | Maltodextrin/Gum acacia / | | for encapsulation |
| | Lactose/ Dextrans | | |

Composition 11

| SI. | | Quantity | |
|---|---|---|---|
| No. | Ingredients | % range | Function |
| 1 | Tea / Marigold/ | 0.1 to 20% | Active Ingredient |
| 2 | Pomegranate/ Tamarind extract | 0.1 to 20% | Active Ingredient |
| | Carriers/Inactive materials/ | | Fat used for |
| 3 | Hydrogenated palm stearin/ | 20-99% | encapsulation or |
| | Hydrogenated rapeseed oil/ | | water soluble |
| | Other vegetable oil | | carriers used for |
| | Maltodextrin/Gum acacia / | | encapsulation |
| | Lactose/ Dextrans | | |

Composition 12

| SI. | | Quantity | |
|---|---|---|---|
| No. | Ingredients | % range | Function |
| 1 | Cinnamon extract / oil | 0.1 to 20% | Active |
| | | | Ingredient |
| 2 | /tea/marigold extract | 0.1 to 20% | Active |
| | | | Ingredient |
| 3 | Polysorbate/propylene | 1-99% | |
| | glycol/glycerol/lecithin/phosphotidyl | | |
| | choline | | |

Composition 13

| SI. | | Quantity % | |
|---|---|---|---|
| No. | Ingredients | range | Function |
| 1 | Cinnamon extract / oil | 0.1 to 20% | Active |
| | | | Ingredient |
| 2 | Pomegranate/ Tamarind extract | 0.1 to 20% | Active |
| | | | Ingredient |
| 3 | Polysorbate/propylene | 1-99% | |
| | glycol/glycerol/lecithin/phosphotidyl | | |
| | choline | | |

Composition 14

| SI. | | Quantity | |
|---|---|---|---|
| No. | Ingredients | % range | Function |
| 1 | Tea / Marigold/ | 0.1 to 20% | Active |
| | | | Ingredient |
| 2 | Cinnamon extract / oil | 0.1 to 20% | Active |
| | | | Ingredient |
| 3 | Polysorbate/propylene | 1-99% | |
| | glycol/glycerol/lecithin/phosphotidyl | | |
| | choline | | |

### EXAMPLES

### Example 1

### Objective

The overall goal of the project was to determine the efficacy of a composition comprising cinnamon oil, tea extract, and pomegranate extract (CO/TE/PE; 1:1:1) in ameliorating parasite load and shedding, disease development and pathogenesis, and augmentation of animal host protective innate mucosal immunity during Cryptosporidium parvum infection using a transgenic mouse infection model.

### Materials and Methods

Twenty-four (24) 8-week-old male interferon-gamma knockout (IFN-γ KO) mice (B6.129S7-IfngTM1 Ts/J) were purchased and allowed to quarantine and acclimatize for 1 week before the commencement of experiments. There were three treatment groups, with 8 individually housed mice per treatment. The treatments consisted of: Group 1, uninfected-untreated; Group 2, C. parvum-infected-untreated (Control, reconstitution reagent DMSO only); Group 3, infected-composition comprising cinnamon oil, green tea extract, and pomegranate extract (1:1:1) (100 mg/kg). Each mouse from the infected groups was infected by oral gavage administration of 104 C. parvum AUCP-1 isolate oocysts on Day 3 after the experiment started.

### Materials and Methods

### 1. Parasites

The Cryptosporidium parvum AUCP-1 isolate was maintained and propagated in male Holstein calves. C. parvum oocysts were extracted and purified from freshly collected calf feces by sequential sieve filtration, Sheather's sugar flotation, and discontinuous sucrose density gradient centrifugation (Arrowood & Sterling, 1987; Current, 1990). Purified oocysts were washed and stored in phosphate-buffered saline (PBS) at 4° C. and used within 3 months to ensure maximum viability as judged by excystation.

### 2. Animals

Male IFN-γ knockout mice (B6.129S7-Ifngtm1 Ts/J), aged 7 weeks, were procured from The Jackson Laboratory, USA, and housed under Biosafety level 2 conditions. Feed (commercial mouse chow) and water were supplied ad libitum. Animals were left to acclimatize for 4 days before the start of experiments.

### 3. Compound

CO/TE/PE was reconstituted in molecular grade dimethyl sulfoxide (DMSO) followed by filter-sterilization using a 0.22μ filter, and stored at room temperature in the dark.

4. Treatment, infection, sample collection and determination of physical parameters Mice were divided into three groups with 8 mice per group. Within each group, each individual mouse was housed in an individual cage. The groups were as follows:
Uninfected untreated group (UU).
Infected DMSO-treated group (DMSO).
Infected CO/TE/PE-treated group

Three days prior to infection with C. parvum, mice in the various treatment groups begun receiving daily oral gavage administration of compounds or DMSO as follows: CO/TE/PE=100 mg/kg; DMSO=0.05 ml DMSO; UU=0.05 ml DMSO. On the third day after commencement of treatment, mice in the infected groups (DMSO, CO/TE/PE) each received 104 C. parvum oocysts suspension in PBS by oral gavage, and the respective daily treatments per group continued for 14 days post-infection. Alongside compounds treatment, mice were provided regular commercial chow diet and water ad libitum. During the entire period of treatment, fecal samples were collected daily, and physical fecal consistence was determined.

Animals were also scored for physical state and activity, body weight and appetite. Moreover, mice were sacrificed and their distal small intestines used for histopathological examination.

### Results

### 1. Physical Parameters:

Generally, the Uninfected Untreated (UU) mice were active, healthy, with good appetite throughout and gained weight. The weight gain and activity observed is typical of healthy mice at this age (7 weeks), as they would still be in growth phase. The Infected DMSO-treated (DMSO) depicted typical cryptosporidiosis disease progression, with diarrhea, weight loss, poor body condition and reduced activity. Overall, they lost weight over the experimental period despite that that they were still growing.

The infected-CO/TE/PE-treated mice had better physical outcomes than the DMSO-group. They did not develop diarrhea, they remained active and healthy looking throughout, and they gained weight, though not as much as the uninfected mice did. At post-mortem, they had healthy-looking intestines, without hemorrhages. From these observations, it was evident that treatment with CO/TE/PE ameliorated cryptosporidiosis and improved the health of the infected animals.

2. Effect of Compound Treatment on C. parvum Oocysts Load in Infected Mice Feces:

As expected, the Infected DMSO-treated control group mice showed a progressive increase in oocysts shedding, with the first a peak load of about 2×108 oocysts per gram of feces (OPG) being attained by day 10 post-infection, with a second wave higher peak of 2.2× 108 oocysts being observed at 15 days post-infection. Treatment with CO/TE/PE depicted oocysts loads that were consistently significantly (P<0.05) lower than those of the infected DMSO-treated mice oocysts throughout the treatment period. These results corroborated the physical parameters that indicated that CO/TE/PE treatment ameliorated disease progression and outcome.

### 3. Amelioration of Histopathological Lesions:

At the end of the treatments, mice were sacrificed and their distal small intestines used for histopathological examination. As expected, the uninfected control group mice showed healthy intestinal mucosa with prominent villi. On the other hand, the infected DMSO-treated mice had intestinal lesions characterized by mucosal erosion, villous atrophy, hypertrophy of the crypts, and accumulation of inflammatory cells. Mice treated with CO/TE/PE showed better outcomes than the infected DMSO-treated mice, depicting mostly intact mucosa with prominent villi.

While CO/TE/PE-treated mice showed accumulation of inflammatory cells in the mucosa, there was no evident hypertrophy of intestinal crypts, indicating that these mice still had the ability to replenish their intestinal epithelial cells from the intact crypts. These findings are consistent with the significantly reduced number of oocysts detected in CO/TE/PE-treated mice, and thus indicate that this treatment ameliorated parasite proliferation and intestinal pathology in the infected mice.

### Example 2

The goal of this experiment was to determine the efficacy of a composition comprising cinnamon oil, tea extract and pomegranate extract (CO/TE/PE; 1:1:1) in ameliorating parasite load and shedding, disease development and pathogenesis, and augmentation of animal host protective innate mucosal immunity during Cryptosporidium parvum infection using a bovine calf infection model.

### Experimental Approach

### Determination of Efficacy in Ameliorating Cryptosporidiosis

The CO/TE/PE-composition was individually administered to calves by oral gavage once daily for 3 days prior to infection, and continued for 14 days post-infection with C. parvum. Alongside compounds treatment, calves were provided a milk-replacer diet and water ad libitum. During the entire period of treatment, fecal samples were collected daily for measurement of C. parvum oocysts shedding by quantitative real-time PCR, and physical fecal consistence determination. Animals were also scored for physical state and activity, body weight, feeding, body temperature, fur condition and body posture. On day 12 post-infection, blood was collected from calves for liver function tests followed by euthanasia of the calves and resection of the distal small intestines. A portion of resected small intestine were used for histological examination of the intestinal epithelium to determine pathology associated with C. parvum. Another portion of the small intestines was used for tissue protein extraction and the extracts were used in an ELISA assay for analyzing the levels of interferon-18 (IL-18) expressed in intestinal mucosal cells as a measure of the anti-cryptosporidial protective innate immune responses. Note: IL-18 activates the release of antimicrobial peptides that can kill C. parvum in intestinal mucosal cells.

The experiments consisted of 3 groups with 5 calves per group as follows:
5 C. parvum-infected calves treated with the CO/TE/PE composition (200 ppm)
5 C. parvum-infected calves, untreated
5 uninfected, untreated calves

### RESULTS AND CONCLUSION

Calves in C. parvum-infected-untreated group had severe diarrhea throughout the study, with 2 animals dying 10 days after infection. On average, the calves in this group lost appr. 14 kg of body weight. They also had poor appetite and physical state and were dehydrated. Calves treated with CO/TE/PE composition showed mild diarrhea and recovered completely before 10 DPI. They lost about 3 kg of BW. The other physical parameters including hydration status were all improved compared to the C. parvum-infected-untreated group. The CO/TE/PE composition effectively mitigated the Cryptosporidiosis symptoms including diarrhea and body weight loss caused by infection with C. parvum.

1. A method of feeding a pre-ruminant or ruminant animal, the method comprising administering to the animal a composition comprising:
(i) cinnamon extract or oil,
(ii) tea extract, and
(iii) pomegranate extract.

2. The method according to claim 1, wherein the composition is comprised in an aqueous solution.

3. The method according to claim 2, wherein the aqueous solution is drinking water or a milk replacer.

4. The method according to claim 1, wherein the composition is comprised in a feed composition.

5. The method according to claim 1, wherein the animal is a calf, a dairy cow, or beef cow.

6. The method according to claim 1, which prevents, reduces the severity of, and/or treats parasitic infections in the pre-ruminant and/or ruminant animal.

7. The method according to claim 6, which prevents body weight loss due to the infection.

8. The method according to claim 1, which prevents, reduces the severity of, and/or treats diarrhea in the pre-ruminant and/or ruminant animal.

9. The method according to claim 8, wherein the diarrhea is caused by an infection, optionally a parasitic infection.

10. The method according to claim 9, wherein the parasitic infection is cryptosporidiosis.

11. A composition, comprising, based on dry matter content:
(i) 15-30 wt % fats;
(ii) 15-35 wt % milk protein;
(iii) 35-55 wt % sugars, optionally comprising lactose; and
(iv) cinnamon extract or oil, tea extract, and pomegranate extract.

12. The composition according to claim 11, which is in powder form.

13. The composition according to claim 11, comprising, based on dry matter content:
(a) less than about 5 wt %, starch; and/or
(b) less than about 10 wt % ash.

14. The composition according to claim 11, wherein the fats comprise vegetable fats.

15. The composition according to claim 14, wherein the vegetable fats comprise palm oil, coconut oil, palm kernel oil, rapeseed oil, linseed oil, sunflower oil, or MCT oil.

16. The composition according to claim 11, wherein the milk protein comprises casein.

17. The composition according to claim 11, wherein the milk protein comprises one or more of beta-lactoglobulin, alpha-lactalbumin, blood serum albumin, immunoglobulins, lactoferrin, and transferrin.

18. The composition according to claim 11, wherein the sugars comprise lactose.

19. A method of reducing oocyst shedding in a subject, the method comprising administering to the subject a composition comprising cinnamon extract or oil, tea extract and pomegranate extract.
